(19) United States
(12) Patent Application Publication
(10) Pub. No.: US 2012/0109251 A1
(43) Pub. Date: May 3, 2012

(54) TRANSCRANIAL ELECTROSTIMULATION DEVICE

(76) Inventors: VALERY PAVLOVICH LEBEDEV, ST. PETERSBURG (RU); ALEKSANDR VYACHESLAVOVICH MALYGIN, LENINGRADSKAYA OBL. (RU)

(21) Appl. No.: 13/299,025

(22) Filed: Nov. 17, 2011

Related U.S. Application Data
(63) Continuation-in-part of application No. 12/297,171, filed on Oct. 14, 2008.

(30) Foreign Application Priority Data
Mar. 23, 2006 (RU) 2006109133

(51) Int. Cl. A61N 1/36

(52) U.S. Cl. 607/45

(57) ABSTRACT

The invention relates to a transcranial electrostimulation device for treating different illnesses. In an exemplary embodiment, the transcranial electrostimulation device includes a bipolar pulse source for generating an electrical current and a patient physiological status processor that provides a biofeedback mechanism for adjusting the applied electrical stimulation. The device further includes electrodes positioned on a patient's skin at a retro-mastoidal location for delivering bipolar electrical pulses to induce transcranial electrostimulation.

### BACKGROUND OF THE INVENTION

1. Field of the Invention

The invention concerns to physiotherapy and is intended for stimulation of protective mechanisms of the brain at the treatment of various diseases.

2. Description of the Related Technology

Conventional devices for transcranial electrostimulation contain a power unit, a current regulator, a current stabilizer, a generator, an amplifier, a current measuring gauge and electrodes. The power unit includes a battery, a power voltage converter, a comparator, an indicator of a battery voltage and a voltage stabilizer. Such devices may also include as a current stabilizer, a switch and a load equivalent (RU 16826 U1, 2001). These devices, however, are not sufficiently effective and safe for administering electrostimulation to a patient.

### SUMMARY OF THE INVENTION

The invention is directed to a safe and highly efficient transcranial electrostimulation device for inducing transcranial electrostimulation to treat patients. Biofeedback channels increase treatment efficiency, allowing a patient's physiological condition to influence the amount of applied electrostimulation. Depending on a patient's physiological status at the time of treatment, a procedure counter monitors and limits the amount of electrostimulation applied to a patient. The amount of electrostimulation is also regulated by a patient identification means for identifying, displaying and storing an individual's patient history and treatment information. This allows both the patient and the doctor to customize a patient's treatment, thereby increasing treatment efficiency and safety.

The transcranial electrostimulation device of the present invention contains a bipolar pulse source for generating and applying a bipolar electrical pulse to treat a patient. In an exemplary embodiment, the bipolar pulse source includes a generator, a current stabilizer, an amplifier and a current measuring gauge. Electrodes operatively associated with the bipolar pulse source are positioned on a patient at a retro-mastoidal location for delivering the electrical pulses. The device further includes a patient physiological status processor, a current regulator, a timer, a memory device, a patient identification means and an information display panel. The generator, the current stabilizer, the amplifier, the current measuring gauge and the electrodes are connected in series. A first output of the patient's physiological status processor is connected to an input of the generator, and its second output is connected to a first input of the current regulator. A second input of the current regulator is connected to an output of the memory device, and a third input of the current regulator is connected to the timer output. An output of the current regulator is connected to the second input of the amplifier. The first and the second inputs of the memory device are respectively connected to an output of the patient identification means and an output of the procedure counter. The second output of the procedures counter may be connected to the first input of the information display panel. The second input of the information display panel is connected to an output of the current measuring gauge, and the third input of the information display panel is connected to the memory device output.

The patient's physiological status processor contains a skin resistance gauge, a cardiac signal gauge, a temperature gauge, a biosignal amplifier, an analog-to-digital converter (ADC) and a data processor The skin resistance gauge, the cardiac signal gauge and the temperature gauge are connected, to the first, second and third inputs of the biosignal amplifier, respectively. The output of the biosignal amplifier is connected to the input of the ADC, which is connected to the data processor.

The memory device includes a memory decoder, a memory storage unit for storing patient specific information, external memory modules and a data transferal device. The memory decoder is connected to the memory storage unit; these components are operatively associated with the data transferal device. The external memory modules are connected to an input of the data transferal device.

### BRIEF DESCRIPTION OF DRAWINGS

FIG. 1 is a schematic diagram of an exemplary embodiment of the electrostimulation device.

FIG. 2 is a schematic diagram of an exemplary patient physiological status processor.

FIG. 3 is a schematic diagram of an exemplary memory device.

### DETAILED DESCRIPTION OF THE PREFERRED EMBODIMENTS

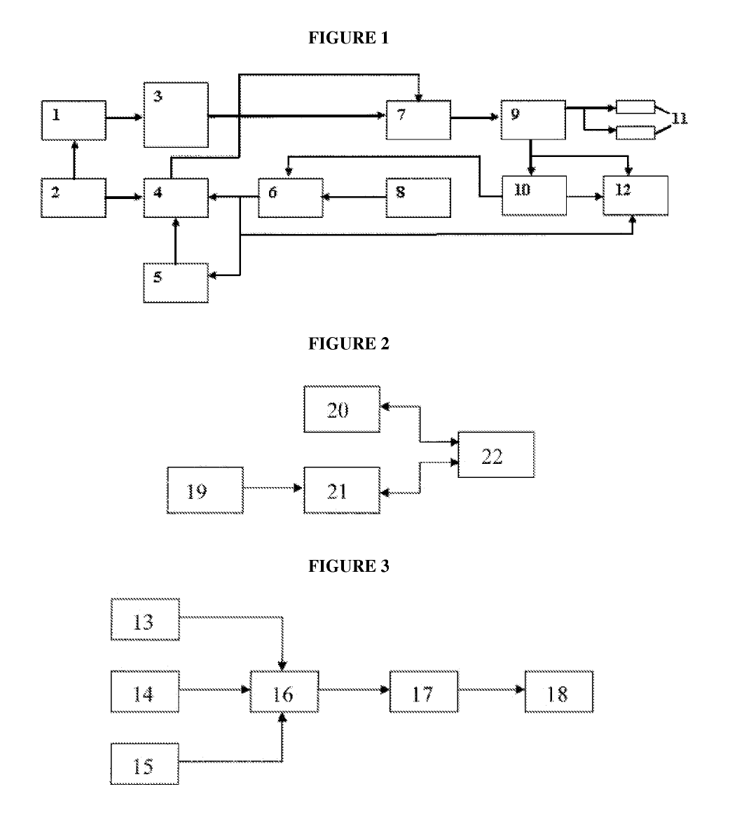

As shown in FIG. 1, the electrostimulation device has a bipolar pulse source that produces bipolar electrical pulses of a predetermined frequency to treat a patient. Bipolar pulse source including a generator 1, a current stabilizer 3, an amplifier 7 and a current measuring gauge 9 that are connected in series. Electrodes 11 are operatively associated with the bipolar pulse source for delivering bipolar electrical pulses for treating a patient. A first output of the patient physiological status processor 2 is operatively associated with the bipolar pulse source, such that it is connected to the generator 1, and the second output is connected to the first input of the current regulator 4. A second input of current regulator 4 is connected to an output of the memory device 6, and a third input of current regulator 4 is connected to the output of timer 5. An output of current regulator 4 is connected to an input of the amplifier 7. The first and the second inputs of the memory device 6 are respectively connected to an output of the patient identification means 8 and an output of the procedures counter 10. An output of memory device 6 is connected to the input of timer 5. A second output of the procedures counter 10 is connected to the first input of the information display panel 12. A second input of information display panel 12 is connected to the output of the current measuring gauge 9, and a third input of information display panel 12 is connected to the output of the memory device 6.

The patient's physiological status processor 2 functions to obtain physiological data from a patient and is operatively associated with the bipolar pulse source so as to adjust the bipolar electrical pulses based on the physiological data obtained from the patient by the patient physiological status processor 2. The patient's physiological status processor 2 includes one or more sensors, such as a skin resistance gauge 13, a cardiac signal gauge 14 and a temperature gauge 15, a biosignal amplifier 16, an ADC 17 and a data processor 18 connected in series to provide biofeedback that gauges the patient's response to the bipolar electrical pulses. The sensors are connected to the first, second and third inputs of the biosignal amplifier 16, respectively. The output of biosignal amplifier 16 is connected to ADC 17, which in turn is connected to the data processor 18.

The memory device 6 includes a memory decoder 19 and a memory storage unit 21 for storing patient specific information; memory decoder 19 and memory storage unit 21 are operatively associated with data transferal device 22. Memory device 6 further includes external memory modules 20, which are connected data transferal device 22.

The operation of the electrostimulation device is described below. After the device is turned on, the patient identification means 8, which contains patient specific information, is first activated. A signal generated by patient identification means 8 is processed by an operatively associated memory device 6. Specifically, patient specific data concerning the amount of electrostimulation procedures previously performed on the patient as well as data concerning customized operating modes for a specific patient are processed. The signal is then sent to and processed by current regulator 4, timer 5 and information display panel 12. Current regulator 4, which is operatively associated with memory device 6 and connected to amplifier 7, then affects and determines the intensity of the generated bipolar electrical pulse. Timer 5, which is connected to current regulator 4, affects and determines the duration of the electrostimulation procedure, e.g. the duration that the bipolar electrical pulse is applied to the patient. The generator 1 preferably produces bipolar electrical pulses having a frequency of 72-85 Hz. The duration of a positive pulse is 3.7±0.5 ms. and the duration of a negative pulse is 9.2±0.5 ms, wherein the treatment produces a zero average current for the period. The bipolar electrical pulses produced by generator 1 are subsequently processed by current stabilizer 3. The parameters of the bipolar electrical pulses may vary during the procedure and depend upon the data received by generator 1 from the patient physiological status processor 2. The signal from patient physiological status processor 2 also affects current regulator 4 and determines the amount, intensity and duration of electrostimulation that may be applied to a specific patient. After being processed by current stabilizer 3 the bipolar electrical current is amplified by the amplifier 7 up to a preset level and subsequently passes through current measuring gauge 9 to the electrodes 11, which are positioned on a patient's skin at a retro-mastoidal location to enable the transcranial delivery of bipolar electrical pulses and induce transcranial electrostimulation so as to treat a patient. The procedure counter 10, which is connected to current measuring gauge 9, generates a signal if the applied bipolar electrical pulse exceeds 0.2 mA for least 15 minutes, indicating the performance of a bipolar electrical pulse treatment. Procedure counter 10 is capable tracking the number of patient administered electrostimulation procedures lasting at least 15 minutes wherein the applied bipolar electrical pulse exceeds 0.2 mA. Procedure counter 10 also counts the number of electrostimulation procedures administered to a specific patient and the total number of the procedures performed by the device. Procedure counter 10 prevents the device from administering more than 50 electrostimulation procedures to a single patient within a calendar year. Procedure counter 10 as well as information from patient identification means 8 may be used to restrict the number and amount of individual transcranial electrostimulation treatment procedures administered to a patient for a predetermined period of time. An individual's patient history and treatment information may be viewed on information display panel 12, allowing a physician to supervise both the course of current procedure, and record the physiological data of the patient. Information display panel 12 is operatively associated with the procedure counter 10 and may display information related to the number of administered electrostimulation procedures.

The operation of the patient physiological status processor 2 is described below. Signals from one or more sensors, such as skin resistance gauges 13, cardiac signal gauges 14 and temperature gauges 15, go to the inputs of the biosignal amplifier 16. After these signals are amplified and converted by the ADC 17, they are fed to data processor 18, wherein the signals are analyzed based on known dependences of the treatment parameters and on the established limits of norms and pathology. Data processor 18 then signals current regulator 4 and the generator 1 to adjust the bipolar electrical current based on the biofeedback obtained from the sensors.

The operation of the memory device 6 is described below. Before the beginning of the treatment procedure, data from the patient identification means 8 is processed by memory device 6, wherein memory decoder 19 produces a signal allowing it to select and access one or more of the cells in the memory storage unit 21. The information contained in a specific cell is then processed by data transferal device 22 and subsequently sent to current regulator 4, timer 5 and procedure counter 10. During treatment, information is also recorded by memory device 6, whereby patient data is conveyed by data transferal device 22 and recorded by memory storage unit 21. Moreover, external memory modules 20 may enable access and transfer of various types of data located on external media or computers. This information may be transferred to current regulator 4, timer 5 and procedure counter 10 via data transferal device 22 for adjusting the bipolar electrical pulses. Additionally, data from the device can be transferred bidirectionally to an external media source or to a computer.

The device provides many means for acquiring, storing, visualizing and using patient data, including data regarding a patient's physiological state during treatment, that may be used to adjust the parameters of the bipolar electrical pulses applied during treatment. Moreover, the procedure counter enables the device to count and track the number of treatments administered to a specific patient and to limit the maximum number of treatments that may be administered to a patient in a calendar year. The invention therefore provides a highly effective and safe electrostimulation device for treating a patient.

1. A transcranial electrostimulation device comprising:
a bipolar pulse source that produces bipolar electrical pulses of a predetermined frequency to treat a patient;
at least two electrodes operatively associated with said bipolar pulse source, wherein said electrodes are positioned on a patient at a retro-mastoidal location so as to deliver the bipolar electrical pulses and induce transcranial electrostimulation; and
a patient physiological status processor that obtains physiological data from the patient, wherein the patient physiological status processor is operatively associated with the bipolar pulse source so as to adjust the bipolar electrical pulses based on the physiological data obtained by the patient physiological status processor.

2. A transcranial electro stimulation device according to claim 1, wherein the bipolar pulse source comprises a generator, a current stabilizer, an amplifier and a current measuring gauge that are connected in series.

3. A transcranial electro stimulation device according to claim 1, wherein said patient physiological status processor comprises a sensor, a signal amplifier, an analog-to-digital converter and a data processor that are connected in series to provide biofeedback that gauges the patient's response to the bipolar electrical pulses, and wherein the sensor may be selected from the group consisting of: a skin resistance gauge, a cardiac signal gauge and a temperature gauge.

4. A transcranial electrostimulation device according to claim 1, further comprising:
a procedure counter that tracks the number of electrostimulation procedures administered to the patient;
an information display panel that displays information regarding the patient or the electrostimulation procedure, wherein the information display panel is operatively associated with the procedure counter;
a patient identification means containing patient specific data;
a memory device operatively associated with the patient identification means and the procedure counter;
a current regulator operatively associated with the memory device; and
a timer operatively associated with the current regulator, the information display panel and the memory device.

5. A transcranial electrostimulation device according to claim 4, wherein said memory device comprises: a memory decoder, a memory storage unit operatively associated with the memory decoder, and external memory modules for accessing externally located information, and wherein the external memory modules and the memory storage unit are operatively associated with a data transferal device of the memory device through which data can be transferred.

6. A transcranial electro stimulation device according to claim 4, wherein the procedure counter and the patient identification means restrict a number of individual transcranial electrostimulation treatment procedures for a predetermined period.

7. A transcranial electro stimulation device according to claim 6, wherein the patient identification means restricts a number of individual treatment procedures for a particular patient to not more than 50 in a calendar year.

8. A transcranial electro stimulation device according to claim 6, wherein said procedure counter generates a signal indicating performance of a bipolar electrical pulse treatment when a generated bipolar electrical pulsed current exceeds 0.2 MA for at least 15 minutes.

9. A transcranial electrostimulation device according to claim 3, wherein said sensor is a skin resistance gauge.

10. A transcranial electrostimulation device according to claim 3, wherein said sensor is a cardiac signal gauge.

11. A transcranial electrostimulation device according to claim 3, wherein said sensor is a temperature gauge.

12. A transcranial electrostimulation device according to claim 2, wherein the device further comprises:
a procedure counter that tracks the number of electrostimulation procedures administered to the patient;
an information display panel that displays information regarding the patient or the electrostimulation procedure, wherein the information display panel is operatively associated with the procedure counter;
a patient identification means containing patient specific data;
a memory device operatively associated with the patient identification means and the procedure counter;
a current regulator operatively associated with the memory device; and
a timer operatively associated with the current regulator, the information display panel and the memory device,
wherein a first output of the patient physiological status processor is connected to an input of the generator and a second output of the patient physiological status processor is connected to a first input of the current regulator;
a first output of the memory device is connected to a second input of the current regulator; an output of the timer is connected to a third input of the current regulator;
an output of the current regulator is connected to an input of the amplifier;
first outputs of the patient identification means and the procedure counter are connected to first and second inputs of the memory device;
a second output of the procedure counter is connected to a first input of the information display panel;
an output of the current measuring gauge is connected to a second input of the information display panel; and
a second output of the memory block is connected to a third input of the information display panel.

13. A transcranial electrostimulation method comprising the steps of:
attaching the transcranial electrostimulation device of claim 1 to a patient such that the two electrodes are positioned on the patient at the retro-mastoidal location;
delivering the bipolar electrical pulses to the patient so as to induce transcranial electrical stimulation; and
adjusting the bipolar electrical pulses based on the obtained physiological data from the patient physiological status processor.

14. A transcranial electrostimulation method according to claim 13, wherein the transcranial electrostimulation device further comprises:
a procedure counter that tracks the number of electrostimulation procedures administered to the patient; and
a patient identification means; and
wherein the method further comprises the step of restricting an amount of bipolar electrical pulse treatments that is administered for a predetermined period.
